(19) United States
(12) Patent Application Publication
(10) Pub. No.: US 2023/0190233 A1
(43) Pub. Date: Jun. 22, 2023

(54) VISUALIZATION OF CHANGE IN ANATOMICAL SLOPE USING 4D ULTRASOUND CATHETER

(71) Applicant: Biosense Webster (Israel) Ltd., Yokneam (IL)

(72) Inventors: Andres Claudio Altmann, Haifa (IL); Assaf Govari, Haifa (IL)

(73) Assignee: Biosense Webster (Israel) Ltd., Yokneam (IL)

(21) Appl. No.: 17/555,902

(22) Filed: Dec. 20, 2021

(51) Int. Cl. A61B 8/00; A61B 8/08; A61B 8/12

(52) U.S. Cl. CPC A61B 8/4254; A61B 8/4494; A61B 8/0841; A61B 8/12; A61B 8/461

(57) ABSTRACT

A system includes (a) a catheter for insertion into an organ of a patient, a distal end of the catheter, the distal end including: (i) one or more ultrasound transducers (UT), which are configured to apply ultrasound (US) waves to the organ and to produce one or more US signals indicative of a surface topography of the organ, and (ii) a position sensor, which is configured to produce one or more position signals indicative of one or more respective positions of the distal end inside the organ, and (b) a processor, which is configured, based on the US signals and the position signals, to: (i) produce an anatomical map of the surface topography, and (ii) visualize a change in a slope of the surface topography.

### FIELD OF THE DISCLOSURE

The present disclosure relates generally to medical imaging, and particularly to methods and systems for imaging using a four-dimensional (4D) ultrasound catheter.

### BACKGROUND OF THE DISCLOSURE

Various techniques for imaging morphology of patient organs, such as ultrasound-based visualization, have been published.

For example, U.S. Pat. No. 7,517,318 describes a system and method for imaging a target in a patient's body. The method includes the steps of providing a pre-acquired image of the target and placing a catheter having a position sensor, an ultrasonic imaging sensor and at least one electrode, in the patient's body.

However, there remains a need for imaging techniques that allow physicians to better visualize the morphology of the anatomy of an organ, particularly areas of the organ that may need to be avoided (or targeted) in a procedure, such as, e.g., a tissue fold during a cardiac ablation procedure.

The present disclosure will be more fully understood from the following detailed description of the examples thereof, taken together with the drawings.

### BRIEF DESCRIPTION OF THE DRAWINGS

FIG. 1 is a schematic, pictorial illustration of a catheter-based ultrasound imaging system, in accordance with an example of the present disclosure;

FIG. 2 is a schematic, pictorial illustration of a surface visualization of an ostium of a pulmonary vein (PV) using the system of FIG. 1, in accordance with an example of the present disclosure;

FIG. 3 is a schematic, pictorial illustration of surface topography visualization at multiple sections of the inner wall of the PV, which is presented over an anatomical map of patient heart, in accordance with an example of the present disclosure; and

FIG. 4 is a flow chart that schematically illustrates a method for visualizing one or more changes in slopes of the surface topography in sections intended to undergo tissue ablation, in accordance with an example of the present disclosure.

### DETAILED DESCRIPTION OF EXAMPLES

### Overview

Tissue ablation procedures are used in various medical applications, such as in treating arrhythmias in a patient heart. Tissue ablation procedures typically comprise electro-anatomical (EA) mapping of the heart, and identifying ablation sites based on the EA mapping. Subsequently, inserting an ablation catheter into a cavity having the selected ablation sites, placing ablation electrodes in contact with tissue intended to be ablated, and applying the ablation signals to the tissue intended to be ablated. One example of tissue ablation procedure is pulmonary vein (PV) isolation, in which an annular section of an inner wall of the PV is ablated so as to block EA waves propagating through tissue of the PV, and thereby treating arrhythmia, such as atrial fibrillation, in the patient heart.

Typically, in a PV isolation procedure, a physician moves a suitable catheter, such as a lasso-type catheter having a lasso-shaped distal end along a longitudinal axis of the PV intended to be ablated. When obtaining the desired position (e.g., an ostium of the PV), the physician may use a manipulator of the catheter for expanding the lasso, so as to place the ablation electrodes of the lasso in contact with the surface of a selected inner wall section of the PV. After verifying that all relevant ablation electrodes are in sufficient contact with the tissue intended to be ablated, the processor controls a radiofrequency (RF) generator (or any other energy source) to apply ablation signals to the tissue via the ablation electrodes. A successful PV isolation procedure produces a lesion shaped as a continuous ring along the annular section of the inner wall of the PV. The continuous lesion is intended to block the propagation of EA waves through the tissue of the respective PV.

In some cases, the surface topography of the annular section of the PV inner wall may be rough, e.g., has a tissue fold or a ridge in which the slope of the PV inner wall changes sharply. For example, if in a specific area the tissue changes for every millimeter of movement in the longitudinal axis by about 2 mm in the radial direction. In such cases, when placing the lasso catheter in contact with the tissue of the inner wall, one or more of the ablation electrodes may not have sufficient contact force (or may not have any contact) with the tissue, which may result in discontinuity of the lesion ring and a failure of the PV isolation procedure. Therefore, before ablating the tissue, it is important to carefully survey the surface of the intended ablation site, and in case the surface is not suitable, e.g., an ablation site having a tissue fold, the physician may want to avoid ablating near the tissue fold. Such tissue folds may appear at a visualization of the PV ostium, and the physician may decide to select another ablation site at the PV wall, for example, along the longitudinal axis of the PV, a few millimeters, or centimeters away from the respective cavity (e.g., left atrium) of the patient heart, as will be shown in FIG. 3 below.

Examples of the present disclosure that are described hereinbelow provide improved techniques for visualizing the surface of an intended ablation site for selecting a proper site for performing tissue ablation, such as in PV isolation procedures. In some examples, a system for imaging the surface of an inner wall of a PV comprises a catheter for insertion into an organ of a patient, and a processor.

In some examples, a distal end of the catheter comprises one or more ultrasound transducers (UT), which are configured to apply ultrasound (US) waves to an organ in question (e.g., heart cavity and respective PV), and to produce one or more US signals indicative of the surface topography of the organ in question. The distal end of the catheter comprises a position sensor, which is configured to produce one or more position signals indicative of one or more respective positions of the distal end inside the organ in question.

In some examples, the processor is configured to receive the US signals and the position signals, and to: (i) produce an anatomical map of the surface topography (e.g., of the inner wall of an annular section of the PV), and (ii) visualize one or more changes in the slope of the surface topography. In some cases, the change in slope may be indicative of a tissue fold or a ridge in the inner wall of the annular section of the PV. Note that when using a four-dimensional (4D) ultrasound imaging technique, the processor is configured to produce three-dimensional (3D) ultrasound-based images, which are presented over time corresponding to the locations visited by the distal end of the catheter in the organ in question (e.g., left atrium of the heart and/or a PV).

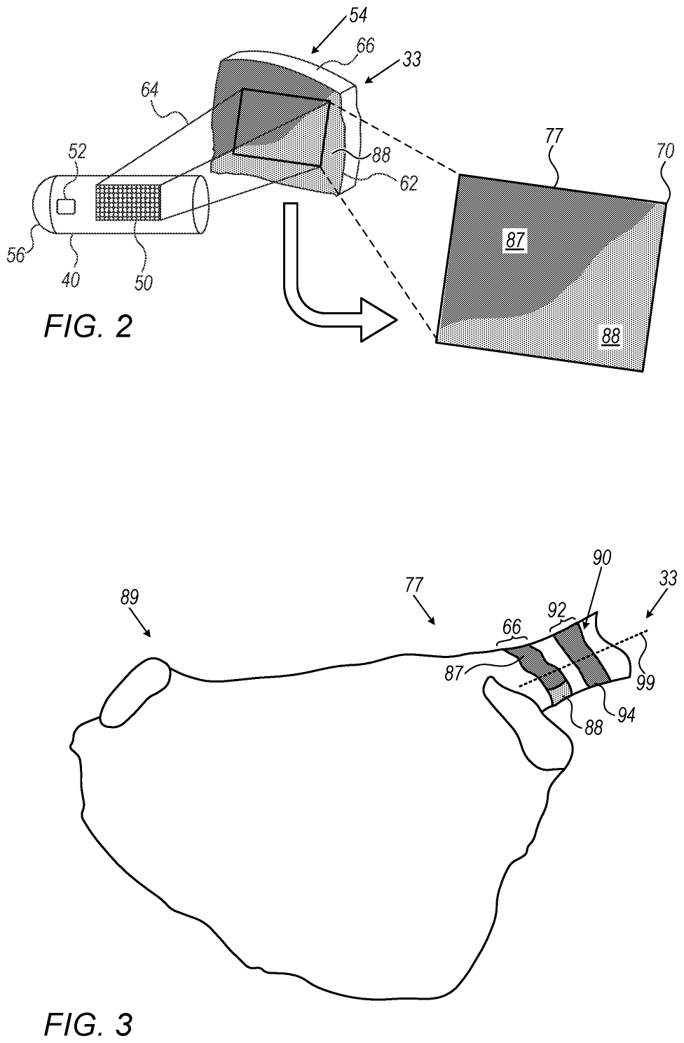

In some examples, the UT are arranged in a two-dimensional (2D) array, and are described in detail in FIG. 2 below. Moreover, the processor is configured to apply to the anatomical map a color-coding scheme indicative of the change in the slope of the surface topography.

In some examples, based on the change in the slope, the processor is configured to detect a fold in an ostium of the PV. Moreover, the processor is configured to determine an alternative annular section of the PV inner wall, which has a smooth surface (without sharp changes in slope), and therefore, is more suitable for applying ablation signals thereto.

In some examples, in addition to visualizing and assessing the quality of the surface of the inner wall, the processor is configured to estimate the thickness at multiple positions along the selected annular section of the inner wall. Based on the estimated thickness, the processor is configured to provide a physician with suitable parameters of ablation signals intended to be applied to the tissue in question. For example, in the selected annular section of the PV, the processor may set a given level of ablation energy to be applied to a first position in which the inner wall is thick, and a level of ablation energy that is lower than the given level of the ablation energy to be applied to a second different position in which the inner wall is thinner.

The disclosed techniques improve the quality of RF ablation procedures by improving the visualization, and hence, the selection of the site intended to be ablated. Moreover, the disclosed techniques may be used, mutatis mutandis, in other sorts of ablation procedures and in any medical procedure that requires a controlled contact force between a medical tool and tissue intended to undergo a treatment or diagnostic procedure.

### System Description

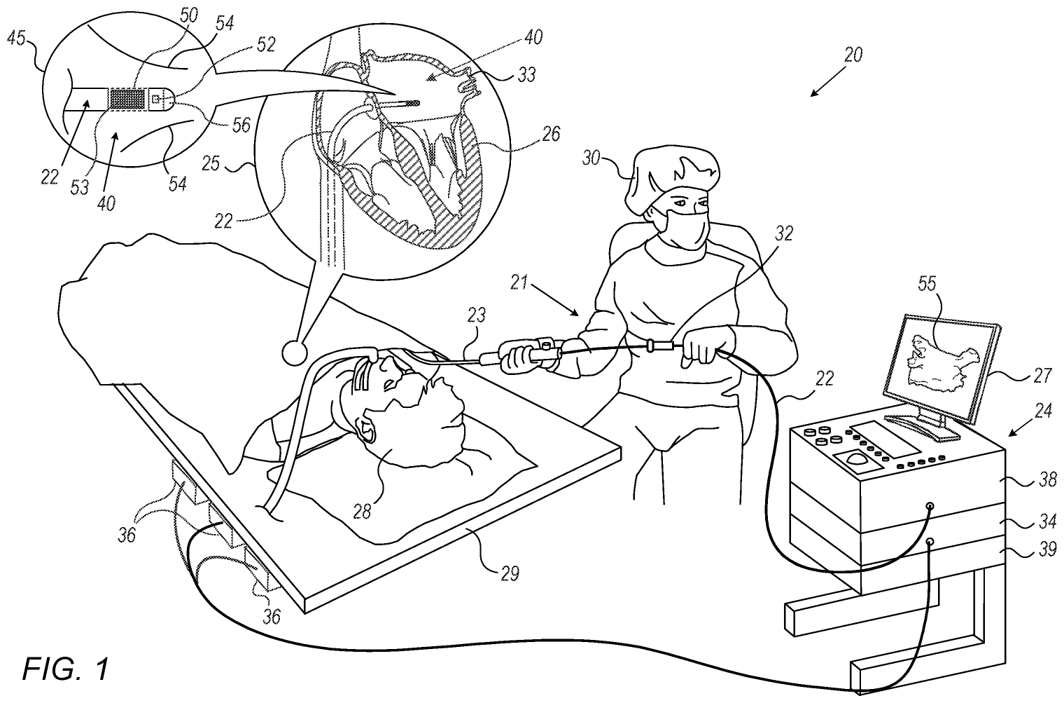

FIG. 1 is a schematic, pictorial illustration of a catheter-based ultrasound imaging system 20, in accordance with an example of the present disclosure.

Reference is now made to an inset 45. In some examples, system 20 comprises a catheter 21 having a distal end assembly 40 that comprises one or more ultrasound transducers (UT) 53 arranged in a two-dimensional (2D) ultrasound array, also referred to herein as a 2D array 50 of the UT, and a position sensor 52.

In some examples, 2D array 50 is configured to apply ultrasound (US) waves to an organ, in the present example, a heart 26 of a patient 28, and to produce one or more US signals indicative of a surface topography and morphology of the respective tissue of heart 26.

In some examples, position sensor 52 is integrated with and is pre-registered with 2D array 50 of catheter 21.

In some examples, position sensor 52 is configured to produce one or more position signals indicative of one or more respective positions of distal end assembly 40 inside heart 26 of patient 28 lying on a surgical table 29, as will be described in more detail herein.

Reference is now made back to the general view of FIG. 1. In some examples, system 20 comprises a processor 39, which is configured, based on the position signals received from position sensor 52, to estimate the direction and the orientation of distal end assembly 40, and more specifically of 2D array 50 of the UT inside (a cavity of) heart 26.

In some examples, based on the position signals received from position sensor 52, processor 39 is configured to produce an US image of heart tissue by registering between ultrasound images that were acquired by 2D array 50, in respective sections of the tissue of heart 26.

In some examples, distal end assembly 40 is fitted at the distal end of a shaft 22 of catheter 21, which is is inserted through a sheath 23 into heart 26. The proximal end of catheter 21 is connected to a control console 24. In the example described herein, catheter 21 is used for ultrasound-based diagnostic procedures, although the catheter may be further used to perform therapy, such as electrical sensing and/or ablation of tissue in heart 26, using, for example, a tip electrode 56 shown in inset 45.

Reference is now made to an inset 25. In some examples, a physician 30 navigates distal end assembly 40 of catheter 21 to a target location in heart 26 by manipulating shaft 22 using a manipulator 32 located near the proximal end of catheter 21. In the example of FIG. 1, physician 30 navigates distal end assembly 40 into a left atrium of heart 26 and applies the UT of 2D array 50, for producing US images of the left atrium and a pulmonary vein (PV) 33 connected to heart 26, both shown in more detail in FIG. 3 below.

Reference is now made back to inset 45. In some examples, before applying radiofrequency (RF) ablation signals to PV 33, physician 30 navigates 2D array 50 into (or in close proximity to) an ostium 54 of PV 33. In some examples, 2D array 50 comprises about 2048 UT 53 arranged in an array of about 64 columns and about 32 rows, and is configured to produce one or more US images of a section of the inner wall of ostium 54. Note that based on the position signals received from position sensor 52, the spatial coordinates of every pixel in the imaged section are known and registered for producing a full US image of the section in question. One implementation of 2D array 50 is shown in FIG. 2 below. Note that the number of UT and the arrangement thereof in 2D array 50 is presented by way of example, and in other examples, 2D array 50 may comprise any other suitable number of UT 53 arranged in any suitable configuration.

In the context of the present disclosure and in the claims, the terms “about” or “approximately” for any numerical values or ranges indicate a suitable dimensional tolerance that allows the part or collection of components to function for its intended purpose as described herein.

Reference is now made back to the general view of FIG. 1. In some examples, control console 24 comprises processor 39, typically a general-purpose computer, with suitable front end and interface circuits 38 for receiving signals from catheter 21, as well as for, optionally, applying treatment via catheter 21 to tissue in heart 26 and for controlling the other components of system 20. Console 24 also comprises a driver circuit 34, configured to drive magnetic field generators 36.

In some examples, during the navigation of distal end assembly 40 in heart 26, console 24 receives position signals from position sensor 52 in response to magnetic fields from external field generators 36. In some examples, magnetic field generators 36 are placed at known positions external to patient 28, e.g., below table 29 upon which the patient is lying. The position signals are indicative of the position and direction of 2D array 50 in the coordinate system of a position tracking system, which is registered with the coordinate system of system 20.

The method of position sensing using external magnetic fields is implemented in various medical applications, for example, in the CARTO™ system, produced by Biosense Webster, and is described in detail in U.S. Pat. Nos. 6,618,612 and 6,332,089, in PCT Patent Publication WO 96/05768, and in U.S. Patent Application Publications 2002/0065455, 2003/0120150, and 2004/0068178.

In some examples, processor 39 is configured to operate 2D array 50 in a “sweeping mode” for fully imaging a cavity (e.g., atrium or ventricle) of heart 26. In an example, processor 39 is configured to present the imaged cavity (e.g., a left atrium) to physician 30 on a monitor 27, e.g., as a volume rendering 55 or using any other presentation.

In some examples, processor 39 typically comprises a general-purpose computer, which is programmed in software to carry out the functions described herein. The software may be downloaded to the computer in electronic form, over a network, for example, or it may, alternatively or additionally, be provided and/or stored on non-transitory tangible media, such as magnetic, optical, or electronic memory.

The example configuration shown in FIG. 1 is chosen by way of example for the sake of conceptual clarity. The disclosed techniques may be applied, mutatis mutandis, using other components and settings of system 20. For example, system 20 may comprise additional components and are configured to perform catheterization procedures other than cardiac.

### Visualization of Tissue Surface Using 4D Ultrasound Catheter

FIG. 2 is a schematic, pictorial illustration of a surface visualization of ostium 54 of PV 33 using system 20 of FIG. 1, in accordance with an example of the present disclosure.

In the context of the present disclosure and in the claims, the term “4D ultrasound” refers to one or more ultrasound transducers, typically arranged in an array, which are configured to apply ultrasound (US) waves to an organ, and to produce one or more US signals indicative of three-dimensional (3D) features of the respective organ, and processor 39 is configured to produce US images based on the US signals. Note that each US image is a 2D image (of a slice of the organ in question) and processor 39 is configured to produce a 3D US image by integrating the 2D slices to a volumetric image having voxels. The fourth dimension is time. When physician 30 moves 2D array 50, processor 39 is configured to produce a video clip comprising the aforementioned 3D US images displayed over time, based on the respective positions of 2D array 50 that is moved within the organ in question.

In the present example, physician 30 intends to perform a PV isolation procedure in order to block electro-anatomical (EA) waves propagating through tissue of PV 33 and thereby, treating arrhythmia in heart 26. In the PV isolation procedure, physician 30 moves a suitable catheter, such as a lasso-type catheter having a lasso-shaped distal end (not shown) along a longitudinal axis of PV 33. When obtaining the desired position, physician 30 may use manipulator 32 for expanding the lasso, so as to place ablation electrodes of the lasso in contact with the surface of an inner wall section 62 of PV 33. After verifying that all relevant ablatipon electrodes are in contact with the tissue intended to be ablated, processor 39 controls a radiofrequency (RF) generator (not shown) to apply ablation signals to the tissue via the ablation electrodes. In the context of the present disclosure, the term inner wall section refers to an annular section that extends along the longitudinal axis of PV 33. One example implementation of PV isolation using a lasso-type catheter is described in U.S. patent application Ser. No. 17/400,414 (attorney docket number BIO6560USNP1 1002-2475).

Note that during the PV isolation procedure, it is important to produce a continuous lesion along the entire annular section of inner wall section 62, by positioning the lasso such that all the ablation electrodes are placed in sufficient contact force with tissue of inner wall section 62. In some cases, a topography in the surface of inner wall section 62 may cause insufficient contact between one or more of the ablation electrodes and the tissue of inner wall section 62. Therefore, it is important to select a suitable position of the lasso when performing the PV isolation procedure. In other words, the surface of the entire annular section of inner wall section 62 must be sufficiently flat, e.g., without sharp changes in the slope of the tissue, so as to ensure sufficient contact force between all the ablation electrodes and the respective tissue of inner wall section 62.

In some examples, before applying the ablation signals, physician 30 inserts distal end assembly 40 into ostium 54 and uses 2D array 50 for applying the US waves and producing one or more US signals indicative of the surface topography of inner wall section 62.

In some examples, the US signals are produced by using 2D array 50 for applying a 3D wedge 64 mode of acquisition that enables simultaneous acquisition of an image of a 2D section 66 of inner wall section 62 of ostium 54. As described in FIG. 1 above, 2D array 50 may comprise about 2048 UT 53 arranged in an array of about 64 columns and about 32 rows or any other suitable configuration. Moreover, 2D array 50 is configured to produce one or more 2D US images of 2D section 66.

In some examples, based on the US signals of 2D array 50 and the corresponding position signals of position sensor 52, processor 39 is configured to visualize the surface of 2D section 66. The method of applying US signals and a position sensor for producing, inter-alia, ultrasound-based anatomical images is described in additional patent applications of the applicant, for example, in U.S. patent application Ser. No. 17/357,231 (attorney docket number BIO6506USNP1/1002-2418) and Ser. No. 17/357,303 (attorney docket number BIO6508USNP1/1002-2439).

Reference is now made to an inset 70. In some examples, processor 39 is configured to produce an image 77 indicative of the surface topography of 2D section 66, and present the image to physician 30 on monitor 27. In the present example, processor 39 is configured to detect that section 66 has a tissue fold, also referred to herein as a fold 88 interfacing with a section 87 of tissue of inner wall section 62. In the context of the present disclosure and in the claims, the term “fold” in the tissue refers to a ridge having one or more sharp changes in the slope of the surface topography, e.g., of section 66. As described above, based on image 77, in case physician 30 will position the lasso in section 66 for applying the ablation signals, the sharp changes in the slope of the surface topography may cause discontinuity of the lesion formed by the ablation, and therefore, to a failure of the PV isolation procedure.

Note that the surface topography of section 87 has a uniform surface without sharp topographical changes. In other words, in the absence of fold 88, the surface of section 87 is sufficiently smooth for placing the ablation electrodes in contact therewith and obtaining a continuous lesion.

### Identifying Suitable Tissue Surface for Performing PV Isolation

FIG. 3 is a schematic, pictorial illustration of surface topography visualization at multiple sections of the inner wall of PV 33, which is presented over an anatomical map 89 of heart 26, in accordance with an example of the present disclosure.

In some examples, processor 39 is configured to present image 77 of FIG. 2, over section 66 of anatomical map 89 of heart 26. As shown in the example of FIG. 3, fold 88 causes a sharp change in the slope of the annular section of the inner wall of section 66. Moreover, in case physician 30 selects section 66 for performing PV isolation, processor 39 is configured to display an alarm, e.g., on monitor 27, so that physician 30 may pursue another site for applying the ablation signals.

In some examples, physician 30 may move distal end assembly 40 along a longitudinal axis 99 of PV 33 and processor 39 is configured to produce a video clip comprising multiple images of respective sections of the inner wall of PV 33. Note that the position of each section is based on the corresponding (i) US signal received from 2D array 50, and (ii) position signal received from position sensor 52.

In some examples, processor 39 is configured to time-gate the visualization of the surface, of each section of the inner wall, responsively to the corresponding cardiac contraction stage of beating heart 26. The time gating is essential for providing physician 30 with the most accurate surface topography of the inner wall of PV 33, without producing imaging artifacts related to different stages of the cardiac contraction of heart 26.

In some examples, processor 39 is configured to present over anatomical map 89 a color-coded motion map indicative of the surface topography of the inner wall of PV 33. In the example of FIG. 3, processor 39 is configured to present over anatomical map 89, an image 90, which is indicative of the surface topography of inner wall tissue 94 of a section 92 of PV 33. Moreover, processor 39 is configured to apply to section 92 a color-coding scheme indicative of the change in the slope of the surface topography of the inner wall. Based on the color-coding scheme, the surface topography of section 92 may appear (e.g., to physician 30) to be suitable for positioning the lasso and applying the ablation pulses to tissue 94. Note that processor 39 is configured to present the entire annular area of section 92, so that physician 30 can verify in advance that section 92 is a suitable site for performing the PV isolation procedure.

In other examples, processor 39 is configured to calculate and display, in any selected section intended to be used for ablation, a metric indicative of one or more changes in slope of tissue within the selected section. Processor 39 is configured to hold one or more thresholds indicative of the allowed level of topography and/or changes in the slope of the tissue within the selected section. In an example, in case the calculated metric exceeds at least one of the thresholds, processor 39 is configured to produce an alert to physician 30.

In the example of FIG. 3, processor 39 is configured to visualize the surface topography of the left atrium (or any other cavity) of heart 26 and PV 33, and of the interface therebetween. In other examples, the disclosed techniques may be used for visualizing the surface topography of any other organs of patient 28. Note that based on the change in slope within section 66, processor 39 is configured to detect fold 88 and to produce an alarm to physician 30.

In some examples, based on the change in slope within section 66, processor 39 is configured to determine an ablation line at one or more locations of PV 33. In the example of FIG. 3, the ablation line is positioned on section 92, which is an annular section of the inner wall of PV 33. As described above, processor 39 is configured to determine the position of the annular section based on the change in the slope of the annular section of the inner wall of PV 33.

In some examples, based on the change in slope within section 66, processor 39 is configured to determine and mark, e.g., on anatomical map 89, a proposed ablation line that is optimized for PV ablation using a focal catheter. The term focal catheter refers to a catheter having one or more ablation electrodes coupled to one position of the catheter. In the example of FIG. 3, processor 39 is configured to mark section 92 over anatomical map 89. The marking may use any suitable user interface features, such as color-coding, textures, or any other suitable feature of graphical user interface (GUI) techniques.

In other examples, based on the US signal(s) from 2D array 50 and the position signal(s) from position sensor 52, processor 39 is configured to estimate the thickness at one or more positions of PV 33, in the present example, the thickness of tissues 87 and 94, and of fold 88. Moreover, based on the thickness at the one or more positions in PV 33 (e.g., positions along section 92), processor 39 is configured to determine one or more parameters of the ablation of tissue 94. For example, in case the thickness of tissue 94 alters with position along section 92, the thicker areas will receive a larger ablation energy compared to that of the thinner areas.

In alternative examples, instead of or in addition to PV 33, anatomical map 89 may comprise organs additional to heart 26, such as but not limited to the esophagus and/or the phrenic nerve of patient 28. In such examples, physician 30 moves distal end assembly 40 in close proximity to the esophagus and/or the phrenic nerve of patient 28, and based on the techniques described above, processor 39 is configured to visualize the surface topography of the respective one or more cavities of heart 26, and of the esophagus and/or the phrenic nerve of patient 28.

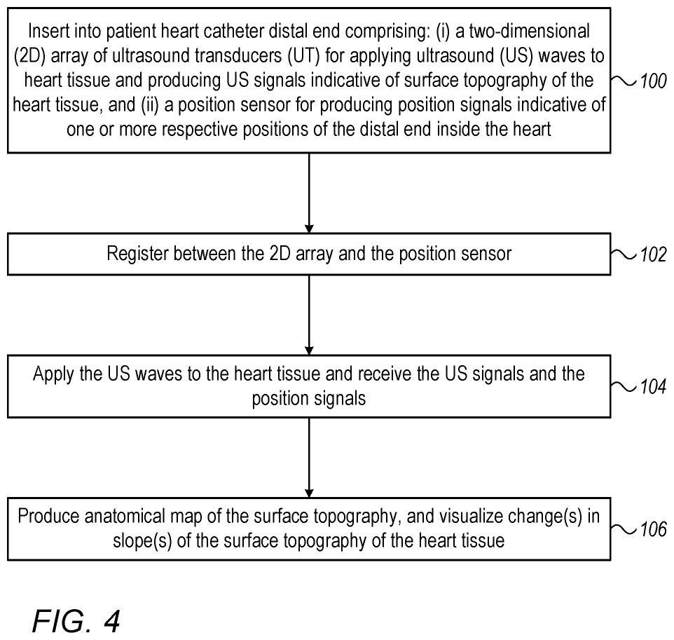

FIG. 4 is a flow chart that schematically illustrates a method for visualizing one or more changes in slopes of the surface topography in sections intended to undergo tissue ablation, in accordance with an example of the present disclosure.

The method begins at a catheter insertion step 100, with inserting distal end assembly 40 into the left atrium of heart 26. As described in FIGS. 1 and 2 above, distal end assembly comprises 2D array 50 of UT and position sensor 52. 2D array 50 is configured to: (i) apply US waves to tissue in question and (ii) produce US signals indicative of the surface topography of the heart tissue in question (e.g., the tissues within sections 66 and 92). Position sensor 52 is configured to produce position signals indicative of the position of distal end assembly 40 inside the left atrium and/or PV 33 of heart 26.

At a registration step 102, the coordinate systems of 2D array 50 and position sensor 52 are registered. Note that typically step 102 is carried out before the catheter insertion for reducing the time of the diagnostic procedure, but in other examples, the registration or verification thereof may be carried out after step 100.

At an ultrasound application step 104, processor 39 controls 2D array 50 to apply the US waves to the tissue of the sections in question, e.g., the tissue of sections 66 and 92. In some examples, processor 39 receives the US signals (from 2D array 50) and the position signals (from position sensor 52), as described in detail in FIGS. 2 and 3 above.

At a visualization step 106 that concludes the method, processor 39 produces anatomical maps of the surface topography of the tissue(s) in question, and presents the produced maps over the respective section(s) (e.g., sections 66 and 92) of anatomical map 89. In some examples, processor 39 visualizes in the maps, change(s) in slope(s) of the surface topography of the tissue(s) in question, so as to assist physician 30 in determining the position for applying the ablation signals to tissue of heart 26 and or PV 33, as shown and described in detail in FIG. 3 above.

The method of FIG. 4 is intended to improve the quality of PV isolation procedures carried out by applying ablation signals to a selected annular section of the inner wall of PV 33. Note that the disclosed components of distal end assembly 40 (e.g., 2D array 50 and position sensor 52) and the method of FIG. 4, may be used, mutatis mutandis, in other applications that require a predefined contact force between a medical tool and tissue of any organ intended to undergo a treatment or a diagnostic procedure.

Moreover, the method of FIG. 4 is simplified for the sake of conceptual clarity, and typically comprises additional steps that are essential to carry out the assessment and visualization of the surface topography and/or the changes in slope within the inner wall of the organ in question.

### EXAMPLE 1

A system (20), including (i) a catheter (21) for insertion into an organ (26) of a patient (28), a distal end (40) of the catheter (21), and (ii) a processor (39). The distal end (40) including: (a) one or more ultrasound transducers (UT) (50), which are configured to apply ultrasound (US) waves to the organ (26) and to produce one or more US signals indicative of a surface topography of the organ (26), and (b) a position sensor (52), which is configured to produce one or more position signals indicative of one or more respective positions of the distal end (40) inside the organ (26). The processor (39) is configured, based on the US signals and the position signals, to: (i) produce an anatomical map (89) of the surface topography, and (ii) visualize a change in a slope of the surface topography.

### EXAMPLE 2

The system according to Example 1, wherein the UT are arranged in a two-dimensional (2D) array.

### EXAMPLE 3

The system according to Example 1 or Example 2, wherein the processor is configured to apply to the anatomical map a color-coding scheme indicative of the change in the slope of the surface topography.

### EXAMPLE 4

The system according to Example 1 or Example 2, wherein the organ includes a cavity in a patient heart and a pulmonary vein (PV), and wherein the processor is configured to visualize the surface topography between the cavity and the PV.

### EXAMPLE 5

The system according to Example 4, wherein, based on the change in the slope, the processor is configured to detect a fold in an ostium of the PV.

### EXAMPLE 6

The system according to Example 4, wherein, based on the change in the slope, the processor is configured to determine an ablation line at one or more locations of the PV.

### EXAMPLE 7

The system according to Example 4, wherein the ablation line includes an annular section of an inner wall of the PV, and wherein the processor is configured to determine a position of the annular section based on the change in the slope of the annular section of the inner wall.

### EXAMPLE 8

The system according to Example 7, wherein the processor is configured to: (i) estimate a thickness at one or more positions of the PV based on the US signals and the position signals, and (ii) determine one or more parameters of an ablation of the PV, based on the thickness at the one or more positions.

### EXAMPLE 9

The system according to Example 1 or Example 2, wherein the organ includes a cavity in a patient heart and an esophagus, and wherein the processor is configured to visualize the surface topography of the cavity and the esophagus.

### EXAMPLE 10

The system according to Example 1 or Example 2, wherein the organ includes a cavity in a patient heart and a phrenic nerve, and wherein the processor is configured to visualize the surface topography of the cavity and the phrenic nerve.

### EXAMPLE 11

A method, including: (a) inserting into an organ (26) of a patient (28), a distal end (40) of a catheter (21) including: (i) one or more ultrasound transducers (UT) (50) for applying ultrasound (US) waves to the organ (26) and producing one or more US signals indicative of a surface topography of the organ (26), and (ii) a position sensor (52) for producing one or more position signals indicative of one or more respective positions of the distal end (21) inside the organ (26), (b) applying the US waves to the organ (26) and receiving the US signals and the position signals, and (c) based on the US signals and the position signals, (i) producing an anatomical map (89) of the surface topography, and (ii) visualizing a change in a slope of the surface topography.

### EXAMPLE 12

The method according to example 11, wherein the UT are arranged in a two-dimensional (2D) array.

### EXAMPLE 13

The method according to Example 11 or Example 12, wherein visualizing the change in the slope of the surface topography includes applying to the anatomical map a color-coding scheme indicative of the change in the slope.

### EXAMPLE 14

The method according to Example 11 or Example 12, wherein the organ includes a cavity in a patient heart and a pulmonary vein (PV), and wherein visualizing the change in the slope of the surface topography includes visualizing the surface topography between the cavity and the PV.

### EXAMPLE 15

The method according to Example 14, and comprising, detecting a fold in an ostium of the PV based on the change in the slope.

Although the examples described herein mainly address tissue ablation and particularly PV isolation, the methods and systems described herein can also be used in other applications, such as in appendage closure and valve replacement.

It will thus be appreciated that the examples described above are cited by way of example, and that the present disclosure is not limited to what has been particularly shown and described hereinabove. Rather, the scope of the present disclosure includes both combinations and sub-combinations of the various features described hereinabove, as well as variations and modifications thereof which would occur to persons skilled in the art upon reading the foregoing description and which are not disclosed in the prior art.

1. A system, comprising:
a processor configured to:
receive, from a catheter having one or more ultrasound transducers, ultrasound signals indicative of a surface topography of an organ of a patient and one or more position signals indicative of one or more respective position of the catheter inside the organ; and
based on the ultrasound signals and the position signals: (i) produce an anatomical map of the surface topography, and (ii) visualize a change in a slope of the surface topography.

2. The system according to claim 1, wherein the one or more ultrasound transducers are arranged in a two-dimensional (2D) array.

3. The system according to claim 1, wherein the processor is configured to apply to the anatomical map a color-coding scheme indicative of the change in the slope of the surface topography.

4. The system according to claim 1, wherein the organ comprises a cavity in a patient heart and a pulmonary vein, and wherein the processor is configured to visualize the surface topography between the cavity and the pulmonary vein.

5. The system according to claim 4, wherein, based on the change in the slope, the processor is configured to detect a fold in an ostium of the pulmonary vein.

6. The system according to claim 4, wherein, based on the change in the slope, the processor is configured to determine an ablation line at one or more locations of the PV.

7. The system according to claim 4, wherein the ablation line comprises an annular section of an inner wall of the PV, and wherein the processor is configured to determine a position of the annular section based on the change in the slope of the annular section of the inner wall.

8. The system according to claim 7, wherein the processor is configured to: (i) estimate a thickness at one or more positions of the pulmonary vein based on the ultrasound signals and the position signals, and (ii) determine one or more parameters of an ablation of the PV, based on the thickness at the one or more positions.

9. The system according to claim 1, wherein the organ comprises a cavity in a patient heart and an esophagus, and wherein the processor is configured to visualize the surface topography of the cavity and the esophagus.

10. The system according to claim 1, wherein the organ comprises a cavity in a patient heart and a phrenic nerve, and wherein the processor is configured to visualize the surface topography of the cavity and the phrenic nerve.

11. A method, comprising:
inserting into an organ of a patient, a distal end of a catheter comprising: (i) one or more ultrasound transducers (ultrasound transducer) for applying ultrasound (ultrasound) waves to the organ and producing one or more ultrasound signals indicative of a surface topography of the organ, and (ii) a position sensor for producing one or more position signals indicative of one or more respective positions of the distal end inside the organ;
applying the ultrasound waves to the organ and receiving the ultrasound signals and the position signals; and
based on the ultrasound signals and the position signals, (i) producing an anatomical map of the surface topography, and (ii) visualizing a change in a slope of the surface topography.

12. The method according to claim 11, wherein the ultrasound transducer are arranged in a two-dimensional (2D) array.

13. The method according to claim 11, wherein visualizing the change in the slope of the surface topography comprises applying to the anatomical map a color-coding scheme indicative of the change in the slope.

14. The method according to claim 11, wherein the organ comprises a cavity in a patient heart and a pulmonary vein (PV), and wherein visualizing the change in the slope of the surface topography comprises visualizing the surface topography between the cavity and the PV.

15. The method according to claim 14, wherein, based on the change in the slope, the processor is configured to detect a fold in an ostium of the PV.

16. The method according to claim 14, wherein visualizing the change in the slope comprises determining an ablation line at one or more locations of the PV.

17. The method according to claim 14, wherein the ablation line comprises an annular section of an inner wall of the PV, and wherein determining the ablation line comprises determining a position of the annular section based on the change in the slope of the annular section of the inner wall.

18. The method according to claim 17, and comprising: (i) estimating a thickness at one or more positions of the pulmonary vein based on the ultrasound signals and the position signals, and (ii) determining one or more parameters of an ablation of the PV, based on the thickness at the one or more positions.

19. The method according to claim 11, wherein the organ comprises a cavity in a patient heart and an esophagus, and wherein visualizing the change in the slope of the surface topography comprises visualizing the surface topography of the cavity and the esophagus.

20. The method according to claim 11, wherein the organ comprises a cavity in a patient heart and a phrenic nerve, and wherein visualizing the change in the slope of the surface topography comprises visualizing the surface topography of the cavity and the phrenic nerve.
